(19) United States
(12) Patent Application Publication
(10) Pub. No.: US 2024/0105326 A1
(43) Pub. Date: Mar. 28, 2024

(54) METHOD AND SYSTEM FOR THE EFFICIENT OPERATION OF LABORATORY MEASURING DEVICES

(71) Applicant: NEDGEX GMBH, Selb (DE)

(72) Inventors: Christian Baier, Hof (DE); Markus Dollinger, Schwarzenbach a. d. Saale (DE); Dominik Meier, Hersbruck (DE)

(21) Appl. No.: 17/769,081

(22) PCT Filed: Oct. 14, 2020
(86) PCT No.: PCT/DE2020/000243

§ 371 Date: Apr. 14, 2022

(30) Foreign Application Priority Data
Oct. 17, 2019 (DE) 102019216033.6

(51) Int. Cl. G16H 40/60; G16H 10/40

(52) U.S. Cl. CPC G16H 40/60; G16H 10/40

(57) ABSTRACT

A computer-implemented method for efficiently operating laboratory measuring equipment (4) of a laboratory measuring equipment operator (LMGB), having the steps of: —receiving (S1) laboratory equipment data that are output by various types of laboratory measuring equipment (4) of the laboratory measuring equipment operator (LMGB) in different output data formats; —converting (S2) the received laboratory equipment data into at least one prescribed target data format of a laboratory information and management system, LIMS, (5) of the laboratory measuring equipment operator (LMGB), wherein data fields of the output data formats are automatically converted to data fields of the target data format in accordance with mapping information stored for the laboratory measuring equipment operator (LMGB); writing (S3) the converted laboratory equipment data in the target data format into a laboratory database (6) of the laboratory information and management system, LIMS, (5) of the laboratory measuring equipment operator (LMGB), wherein the laboratory measuring equipment of the laboratory measuring equipment operator (LMGB) communicates with the laboratory information and management system, LIMS, (5) of the laboratory measuring equipment operator (LMGB) to increase the overall utilization of the laboratory measuring equipment (4) of the laboratory measuring equipment operator (LMGB).

The invention relates to a method and a system for the efficient operation of laboratory measuring devices of an operator of laboratory measuring devices (laboratory measuring device operator, LMGB), in particular an efficient operation of thermoanalytical laboratory measuring devices. The total utilisation of the laboratory measuring devices is increased and the flexibility in the operation of the laboratory measuring equipment is increased.

The term thermal analysis refers to any measuring method, in which a material sample, which may be of known or unknown composition or nature, is subjected to a temperature change in a controlled manner, wherein during this temperature change one or more physical variables on the material sample are simultaneously measured and recorded as a temperature-dependent measurement signal.

Such laboratory measuring devices can be used to determine thermophysical properties of material samples. The physical and chemical properties of materials change under the influence of temperature, atmosphere and pressure. In addition to the examination of thermophysical properties, laboratory measuring devices can be used to track curing, dynamic-mechanical analysis and calorimetry. The laboratory measuring devices thus allow material characterisation as well as determination of specific heat capacity, enthalpy, mass change, modulus of elasticity, thermal expansion and contraction, temperature and thermal conductivity as well as analysis of the gases released, e.g. during thermal decomposition. From this, conclusions can be drawn about the purity and composition of materials, their thermal stability as well as their temperature limits in various applications. Furthermore, ageing processes, thermomechanical behaviour and viscoelastic properties of the materials can be determined. A material sample of the material to be examined is preferably placed for examination purposes into a heating oven, by which the material sample is heated, wherein different types of sensors generate measurement data. The recorded measured variables include e.g. the expansion behaviour, weight changes as well as phase transformation temperatures or enthalpy changes of the measurement sample during the measurement procedure.

There is a large number of different types of laboratory measuring devices which are produced by different laboratory measuring device producers. Therefore, a laboratory measuring device operator typically uses a large number of heterogeneous laboratory measuring devices of the most varied types which are produced by different laboratory measuring device producers, for performing different measurements on one or more material samples. The laboratory measuring devices of a laboratory measuring device operator can be located at a central site or distributed over various sites. The various laboratory measuring devices of a laboratory measuring device operator can be located in a measuring laboratory and/or in the vicinity of a production plant. The measurement samples are taken e.g. in a production process and are supplied to one or more laboratory measuring devices for analysis. The material samples can be taken e.g. manually from the production chain and introduced into the heating oven of the respective laboratory measuring device. In many cases, it is also possible to take the measurement samples automatically in a production stage of the production plant and to introduce them into a heating oven of the laboratory measuring device or test device for carrying out analytical tests. A large number of laboratory measuring devices of a laboratory measuring device operator can provide a large amount of laboratory device data in different data formats. In order to manage and analyse the multitude of laboratory device data obtained, laboratory measuring device operators use laboratory information and management systems (LIMS). These laboratory information and management systems support the processing of the different types of material samples and store the accruing data in a structured, evaluable form in a database. There is a large number of different providers of laboratory information and management systems. Generally, a laboratory measuring device operator uses a laboratory information and management system from a specific provider. LIMS-enabled laboratory measuring devices or instruments can store their laboratory device data as a data set in files so that they can be processed by applications. The laboratory information and management systems of the various providers have import interfaces which support common file formats for the reading-in of structured data. However, laboratory measuring device operators mostly use laboratory measuring devices from different producers and of different types in different variants. In many cases, it is therefore not possible to connect all laboratory measuring devices of a laboratory measuring device operator to the laboratory information and management system (LIMS) used by the laboratory measuring device operator. In many cases, it is also not possible to readily replace a laboratory measuring device of a specific type or producer with another laboratory measuring device of another type or from another producer. Since laboratory measuring devices of different types or from different producers use differently structured data formats, it is also not possible to update the inventory data in the LIMS when a laboratory measuring device is replaced with another laboratory measuring device. This makes it more difficult, if not impossible, to replace a laboratory measuring device with one from another producer. The laboratory measuring device operator which operates a large number of different laboratory measuring devices, thus has very little flexibility to expand their existing fleet of laboratory measuring devices or to replace a laboratory measuring device with another laboratory measuring device from another or new producer. In particular, it is not possible to have industry-wide networking of laboratory measuring devices with the laboratory information and management system (LIMS) used by the laboratory measuring device operator. In the case of laboratory measuring devices of the laboratory measuring device operator which cannot be connected to the laboratory information and management system of the laboratory measuring device operator, it is also necessary to carry out the data management manually. This leads to a significant amount of work and a significant delay in the analysis of the material samples. Owing to the lack of connection of at least some of the laboratory measuring devices of the laboratory measuring device operator, capacity and utilisation planning of the various laboratory measuring devices for examination of different material samples is not possible. This leads to a relatively low utilisation of the various laboratory measuring devices of the laboratory measuring device operator.

Therefore, it is the object of the present invention to provide a method and a system which avoid the aforementioned disadvantages of conventional systems and allow laboratory measuring devices of a laboratory measuring device operator to be operated more efficiently and more flexibly.

In accordance with the invention, this object is achieved by a computer-implemented for method for the efficient operation of laboratory measuring devices having the features stated in claim 1.

According to a first aspect, the invention provides a computer-implemented method for the efficient operation of laboratory measuring devices of a laboratory measuring device operator, comprising the steps of:
receiving laboratory device data which are output by different types of laboratory measuring devices of the laboratory measuring device operator in different output data formats,
converting the received laboratory device data into at least one specified target data format of a laboratory information and management system of the laboratory measuring device operator, wherein data fields of the output data formats are automatically converted or mapped to data fields of the target data format according to mapping information stored for the laboratory measuring device operator,
writing the converted laboratory device data in the target data format to a laboratory database of the laboratory information and management system (LIMS) of the laboratory measuring device operator, wherein the laboratory measuring devices of the laboratory measuring device operator (LMGB) communicate with the laboratory information and management system (LIMS) of the laboratory measuring device operator (LMGB) to increase the total utilisation of the laboratory measuring devices of the laboratory measuring device operator (LMGB).

With the aid of the method in accordance with the invention, it is possible to replace or exchange laboratory measuring devices, so that the flexibility for operating different laboratory measuring devices (different type and/or different producer) is significantly increased. This increases the efficiency during operation of different laboratory measuring devices, i.e. in particular also the number of samples examined per unit of time.

A laboratory information and management system (LIMS) in terms of this present invention can be programs which are developed specifically for this purpose and are designed purely for use in a laboratory or in production control. On the other hand, laboratory information and management systems (LIMS) can also simply be merely databases or any other type of software solutions which are suitable for processing, evaluating or transmitting data. Such programmes can also be e.g. CRM tools, in which customer data are processed on the sales side and linked to measurement data from measuring devices by means of the system in accordance with the invention. This list is not exhaustive and is not intended to limit the invention to specific software solutions.

The focus of the method and system in accordance with the invention is on laboratory measuring devices. They are not intended to limit the invention. It is clear to the person skilled in the art that the method in accordance with the invention and the system in accordance with the invention can also be used in production control or in all areas, in which data and/or measured values from a device have to be converted for a database and/or an evaluation tool.

In one possible embodiment, the respective utilisation of the different types of laboratory measuring devices of the laboratory measuring device operator is determined on the basis of the laboratory device data converted into the target data format of the laboratory information and management system of the laboratory measuring device operator.

In one possible embodiment, the laboratory measuring devices of the laboratory measuring device operator are controlled to increase the total utilisation of the laboratory measuring devices depending on the determined utilisation of the laboratory measuring devices of the laboratory measuring device operator.

In one possible embodiment of the computer-implemented method in accordance with the invention, the laboratory device data of the laboratory measuring device comprise master data of the laboratory measuring device, measurement data of the laboratory measuring device and/or operating status data of the laboratory measuring device.

In a further possible embodiment of the computer-implemented method in accordance with the invention, the different types of laboratory measuring devices of the laboratory measuring device operator comprise laboratory measuring devices of different laboratory measuring device producers and/or different laboratory measuring device types which generate measurement data and output said data in defined output data formats. Since these are different laboratory measuring devices or laboratory measuring device types from different laboratory measuring device producers, the defined output data formats may be different.

In a further possible embodiment of the computer-implemented method in accordance with the invention, the output data formats include structured data sets with data fields for different measurement parameters and/or master data types.

In a further possible embodiment of the computer-implemented method in accordance with the invention, the mapping information of the laboratory measuring device operator is stored in a configurable mapping table which allocates to each data field of an output data format a data field of the target data format of the laboratory information and management system of the laboratory measuring device operator.

In a further possible embodiment of the computer-implemented method in accordance with the invention, during the conversion the laboratory device data contained within a data field of the output data format are imported, in particular copied, into the allocated data field of the target data format of the laboratory information and management system of the laboratory measuring device operator.

In a further possible embodiment of the computer-implemented method in accordance with the invention, the mapping information contained in the mapping table of the laboratory measuring device operator is processed in a configuration routine and/or automatically updated during operation of the laboratory measuring devices.

In a further possible embodiment of the computer-implemented method in accordance with the invention, the laboratory device data of the laboratory measuring device, which are stored in the laboratory database in the target data format of the laboratory information and management system of the laboratory measuring device operator, can be converted back into the output data format in response to a control command for back-conversion according to the mapping information stored in the mapping table of the laboratory measuring device operator.

In a further possible embodiment of the computer-implemented method in accordance with the invention, the output and target data have the following formats: a CSV data format, an INI data format, an XML data format, an ODS data format, an ODT data format, a JSON data format, an RSS data format, an RDF data format, a TXT data format and a TDMS data format. This list is not exhaustive. For the person skilled in the art, it is clear that all known data formats can be converted and mapped and thus made accessible to any laboratory information and management system. The previous list includes only the best-known data formats.

In a further possible embodiment of the computer-implemented method in accordance with the invention, the laboratory measuring devices of the laboratory measuring device operator have material testing devices for performing measurements on material samples, wherein the measurement data generated in the process are output in an output data format via a data interface of the laboratory measuring device.

In a further possible embodiment of the computer-implemented method in accordance with the invention, a controller of the laboratory measuring device of the laboratory measuring device operator receives control commands including measurement specifications for performing measurements on material samples from the laboratory information and management system of the laboratory measuring device operator during an operation.

In a further possible embodiment of the computer-implemented method in accordance with the invention, the laboratory measuring device of the laboratory measuring device operator transmits laboratory device data provided as a client in an output data format of the laboratory measuring device, which are transmitted as user data in data packets via a data network to a local or external (remote) server which executes a server application which automatically converts the received laboratory device data of the laboratory measuring device into the specified target data format of the laboratory information and management system of the laboratory measuring device operator according to the mapping information stored for the laboratory measuring device operator.

In a further possible embodiment of the computer-implemented method in accordance with the invention, when a first laboratory measuring device of a specific laboratory measuring device producer and/or of a specific laboratory measuring device type is replaced by a second laboratory measuring device of another laboratory measuring device producer and/or of another laboratory measuring device type, the laboratory device data of the first laboratory measuring device stored in the laboratory database of the laboratory information and management system of the laboratory measuring device operator in the target data format are seamlessly updated automatically with laboratory device data of the second laboratory measuring device in the target data format.

In a further possible embodiment of the computer-implemented method in accordance with the invention, during an operation of a laboratory measuring device of a laboratory measuring device operator, there is a bidirectional communication link between the laboratory measuring device of the laboratory measuring device operator and the laboratory information and management system of the laboratory measuring device operator for the purpose of exchanging laboratory device data and control commands.

The bidirectional communication link comprises a direct communication link or an indirect communication link via a conversion apparatus.

In a further possible embodiment of the computer-implemented method in accordance with the invention, the laboratory device data and the control commands are exchanged in real time via the bidirectional communication link existing between the laboratory measuring device and the laboratory information and management system.

According to a further aspect, the invention provides a system for the efficient operation of laboratory measuring devices of a laboratory measuring device operator having the features stated in claim 17.

Accordingly, the invention provides a system for the efficient operation of laboratory measuring devices of a laboratory measuring device operator, the system comprising:
laboratory measuring devices of the laboratory measuring device operator, which each provide laboratory device data in different output data formats,
a conversion apparatus for converting the received laboratory device data into at least one specified target data format of a laboratory information and management system of the laboratory measuring device operator,
wherein data fields of the output data formats are automatically converted or mapped to data fields of the target data format according to mapping information stored for the laboratory measuring device operator in a mapping table and the converted laboratory device data in the target data format are written to a laboratory database of the laboratory information and management system of the laboratory measuring device operator, wherein the laboratory measuring devices of the laboratory measuring device operator (LMGB) communicate with the laboratory information and management system (LIMS) of the laboratory measuring device operator (LMGB) in order to increase the total utilisation of the laboratory measuring devices of the laboratory measuring device operator (LMGB).

In one possible embodiment of the system, the laboratory information and management system of the laboratory measuring device operator is used to determine the respective utilisations of different types of laboratory measuring devices on the basis of the laboratory device data converted into the target data format of the laboratory information and management system and written to the laboratory database of the laboratory information and management system, and to control the laboratory measuring devices by means of control commands in order to increase the total utilisation of the laboratory measuring devices of the laboratory measuring device operator, which control commands the laboratory information and management system of the laboratory measuring device operator transmits to the laboratory measuring devices of the laboratory measuring device operator directly or indirectly via a conversion apparatus.

According to a further aspect, the invention further provides a conversion apparatus for automatically converting laboratory device data received in different output data formats into at least one specified target data format of a laboratory information and management system of a laboratory measuring device operator, wherein data fields of the output data formats are automatically mapped to data fields of the target data format according to mapping information stored in a mapping table of the laboratory measuring device operator.

In one possible embodiment, the conversion apparatus is implemented on a cloud server. Furthermore, it is possible that the conversion of the output data is performed inside the laboratory measuring device. In this embodiment of the invention, the laboratory measuring devices of the laboratory measuring device operator can communicate directly with the laboratory information and management system of the laboratory measuring device operator and exchange the necessary data.

The invention also provides a computer program product having stored program commands for performing a method according to the first aspect of the invention.

Possible embodiments of the computer-implemented method in accordance with the invention and of the system for the efficient operation of laboratory measuring devices of a laboratory measuring device operator are explained in greater detail hereinafter with reference to the figures.

In the drawings:

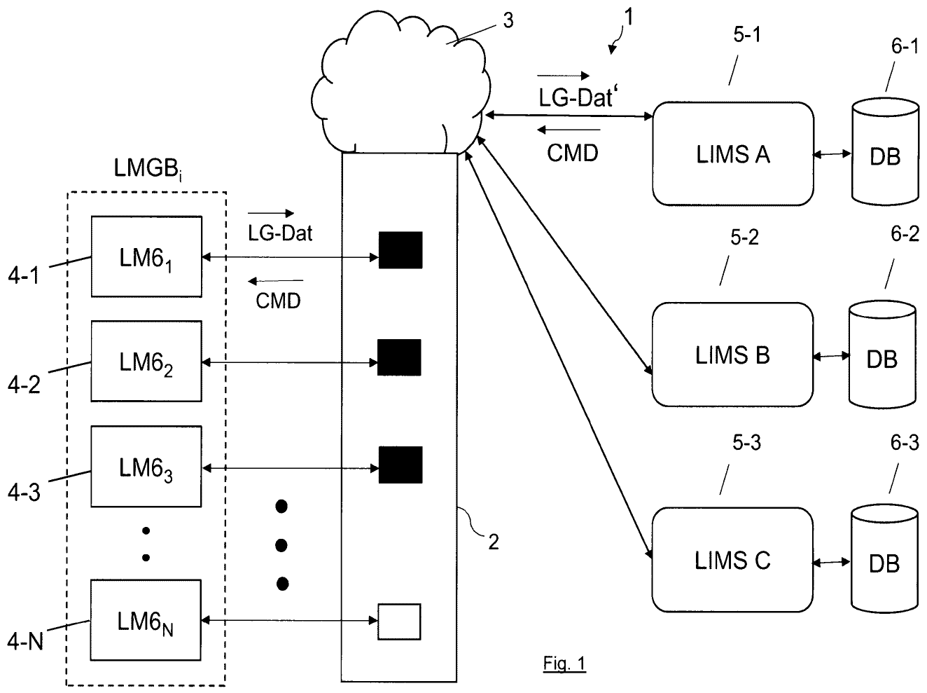

FIG. 1 shows a schematic view of one possible embodiment of the inventive system for the efficient operation of laboratory measuring devices of a laboratory measuring device operator;

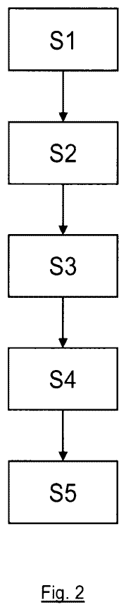

FIG. 2 shows a flow diagram illustrating one possible embodiment of a computer-implemented inventive method for increasing the utilisation of laboratory measuring devices of a laboratory measuring device operator;

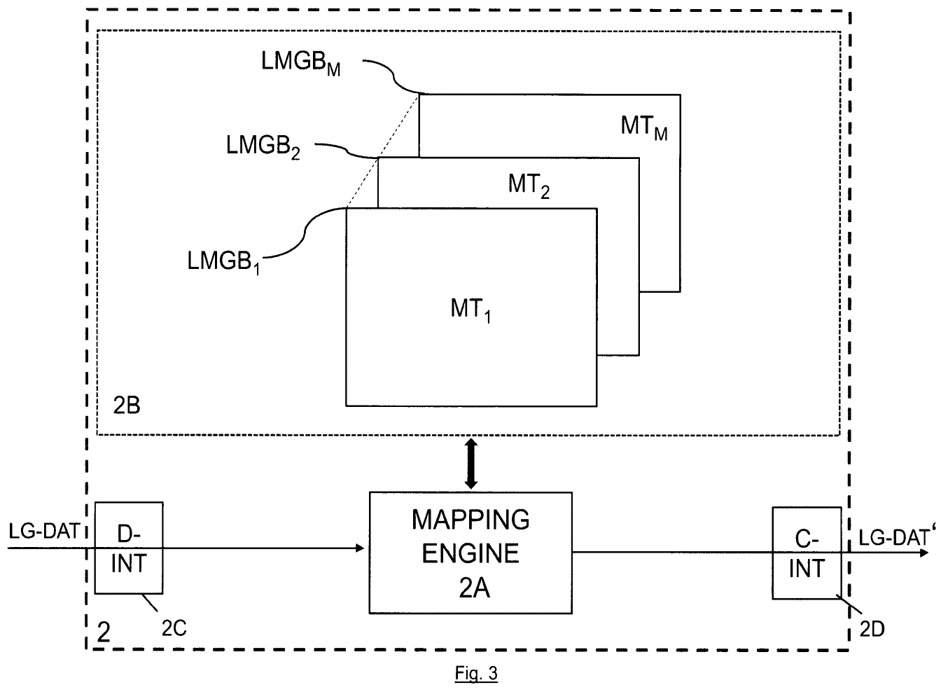

FIG. 3 shows a block diagram of one possible embodiment of a conversion apparatus in accordance with the invention which can be used in the system shown in FIG. 1;

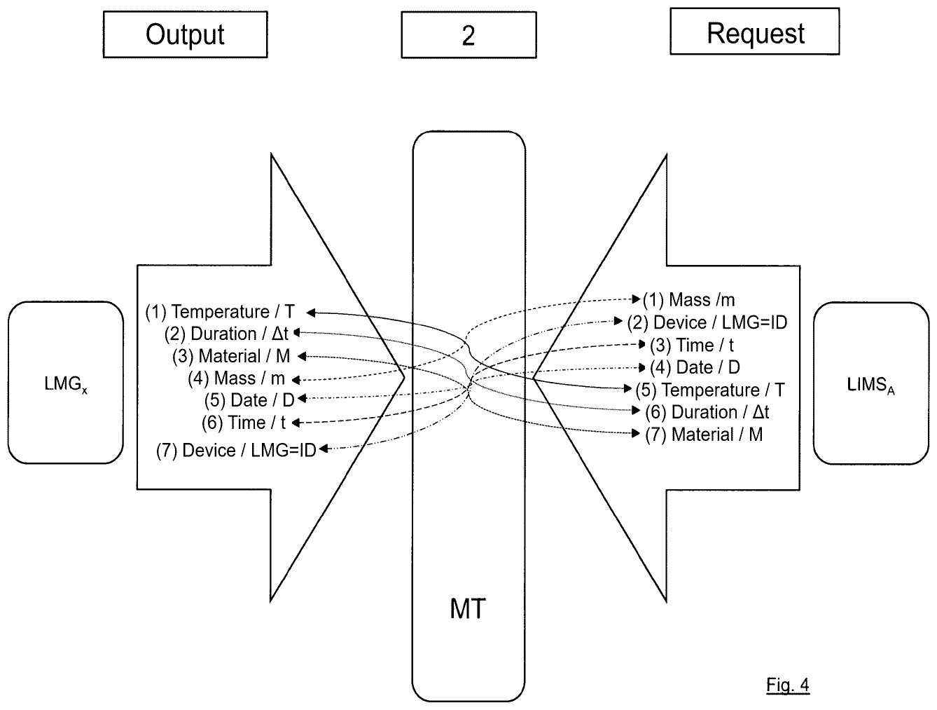

FIG. 4 shows a schematic view explaining the mode of operation of the inventive, computer-implemented method for the efficient operation of laboratory measuring devices of a laboratory measuring device operator;

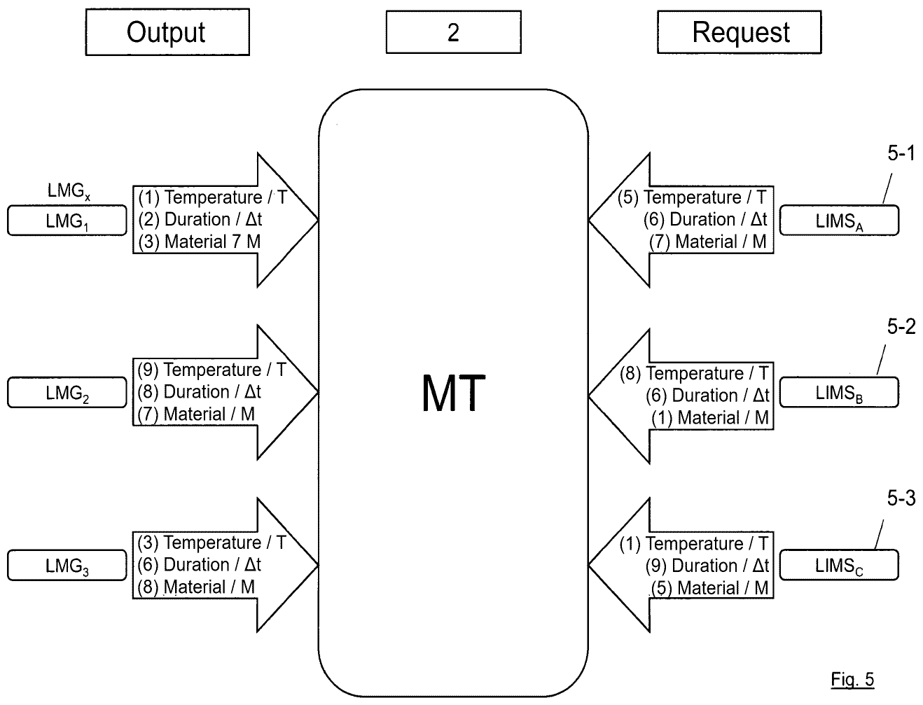

FIG. 5 shows a further schematic view explaining the mode of operation of the computer-implemented method for the efficient operation of laboratory measuring devices of a laboratory measuring device operator;

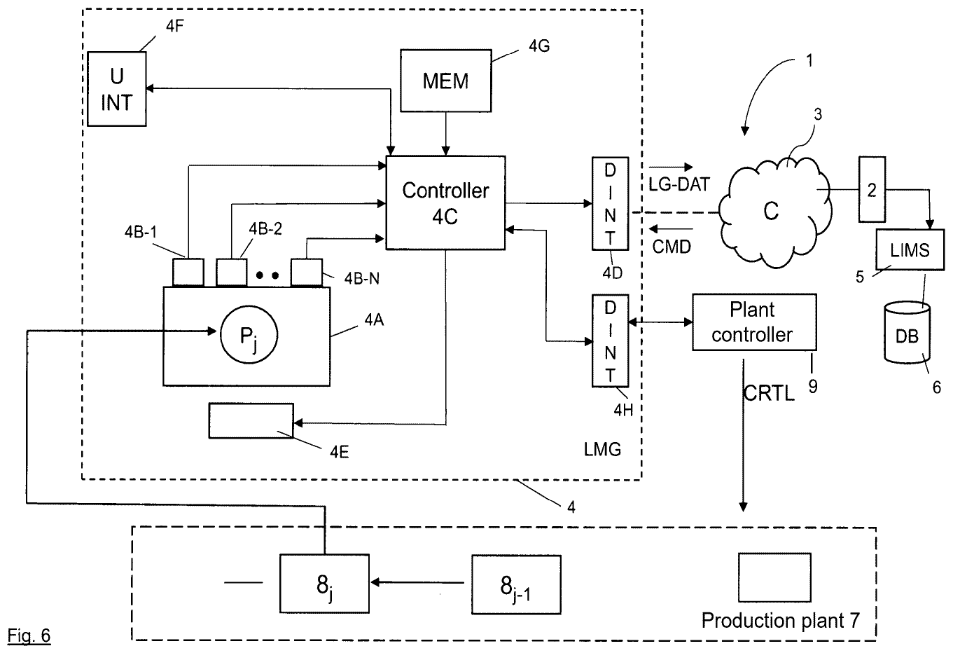

FIG. 6 shows a schematic view of an application example of an inventive, computer-implemented method for the efficient operation of laboratory measuring devices of a laboratory measuring device operator;

As can be seen in FIG. 1, a system 1 in accordance with the invention for the efficient operation of laboratory measuring devices 4 of a laboratory measuring device operator LMGB comprises a conversion apparatus 2 for automatically converting received laboratory device data into a target data format of a laboratory information and management system (LIMS) 5 of the laboratory measuring device operator LMGB. In one possible embodiment, the conversion apparatus 2 of the system 1 in accordance with the invention can be implemented on a server of a data cloud 3. In this case, various laboratory measuring devices 4-1, 4-2, 4-3 . . . 4-n of a laboratory measuring device operator LMGBᵢ each provide laboratory device data of the laboratory measuring device 4 as a client in an output data format of the laboratory measuring device 4. Each laboratory measuring device operator LMGBᵢ can have a unique identifier (LMGB ID). The laboratory measuring devices 4 have e.g. thermoanalytical measuring devices. The conversion apparatus 2 can receive laboratory device data originating from different and different types of laboratory measuring devices 4 of the laboratory measuring device operator in different output data formats, as schematically illustrated in FIG. 1. In one possible embodiment, the conversion apparatus 2 may be implemented on a local or remote server executing a server application. The server application automatically converts the laboratory device data received from a laboratory measuring device 4 in a defined output data format into a target data format of a laboratory information and management system (LIMS) 5 of the laboratory measuring device operator LMGB. The computer-implemented conversion apparatus 2 can receive laboratory device data from various laboratory measuring devices 4 from the same or different laboratory measuring device operators LMGB. Each laboratory measuring device operator LMGB manages its data in a laboratory information and management system LIMS selected by it. In the example illustrated in FIG. 1, a laboratory measuring device operator LMGB can have the laboratory device data originating from its laboratory measuring devices 4 managed and evaluated with the aid of a laboratory information and management system LIMS selected by it. For example, a laboratory measuring device operator LMGB has the choice between a first laboratory information and management system (LIMS_{A}) 5-1, a second laboratory information and management system (LIMS_{B}) 5-2 and a third laboratory information and management system (LIMS_{C}) 5-3, as illustrated schematically in FIG. 1. Each of the laboratory information and management systems LIMS preferably has an associated laboratory database 6. In the system 1 illustrated in FIG. 1, the first laboratory information and management system 5-1, the second laboratory information and management system 5-2 and the third laboratory information and management system 5-3 are connected to the conversion apparatus 2 via the cloud 3 or the data network. In one possible embodiment, the various laboratory information and management systems 5-1, 5-2, 5-3 have associated laboratory databases 6-1, 6-2, 6-3. In one possible embodiment, the conversion apparatus 2 has a microprocessor or an ASIC or an FPGA for executing the mapping.

The conversion apparatus 2 which is implemented on a server in one possible further embodiment is designed according to FIG. 3 in one possible implementation. The conversion apparatus 2 includes a mapping engine 2A and a configuration data memory 2B. The conversion apparatus 2 has a data interface 2C with one or more ports for receiving laboratory measuring device data provided by laboratory measuring devices 4 of the same or different laboratory measuring device operator LMGB. The conversion apparatus 2 can recognise from which laboratory measuring device operator LMGB the received laboratory measuring device data originate. The recognition of the laboratory measuring device operator LMGB occurs in a different way depending upon the embodiment. In one embodiment, the laboratory measuring device operators LMGB are connected via a gateway of the LMGB to an associated port of the conversion apparatus 2, i.e. the conversion apparatus 2 recognises by means of a port number from which LMGB the received laboratory measuring device data originate. Alternatively, received data packets DP which contain laboratory measuring device data as user data can have a data field in their header to indicate the laboratory measuring device operator LMGB (LMGB-ID). Furthermore, in a further embodiment, a source address in the header of the received data packet DP can be used to identify the laboratory measuring device 4 and its laboratory measuring device operator LMGB by means of the conversion apparatus 2. Alternatively, in a handshake procedure, the conversion apparatus 2 can further request the identity of the laboratory measuring device operator LMGB and/or the type of the laboratory measuring device 4, e.g. by means of a corresponding request to a gateway of the laboratory measuring device operator LMGB. The mapping table MT of the recognised laboratory measuring device operator LMGB is used by the mapping engine 2A to convert or reformat the output data format of the received laboratory device data into the target data format of the LIMS of the laboratory measuring device operator LMGB. Furthermore, the conversion apparatus 2 has a cloud connector interface 2D in communication with the cloud 3, as illustrated in FIG. 1. The mapping engine 2A of the conversion apparatus 2 is designed to automatically convert the laboratory device data received in output data formats into a specified target data format of the laboratory information and management system LIMS used by the laboratory measuring device operator LMGB. In one possible embodiment, the LMGB can define a target data format. For this purpose, the mapping engine 2A of the conversion apparatus 2 has access to the configuration data memory 2B, in which an associated mapping table MT is located for each laboratory measuring device operator LMGB. For example, the configuration memory 2B contains M mapping tables MT for M different laboratory measuring devices operators LMGB. Mapping information for each laboratory measuring device operator LMGB is stored in the mapping tables MT. Data fields of the laboratory device data received in the output data formats are automatically mapped or converted by the mapping engine 2A to data fields of the target data format of the laboratory information and management system LIMS used by the laboratory measuring device operator LMGB according to the mapping information stored in the mapping table MT of the respective laboratory measuring device operator LMGB. For this purpose, during the conversion procedure, the laboratory device data contained within a data field of the output data format can be imported, in particular copied, into the allocated data field of the target data format by means of the mapping engine 2A. The laboratory device data include the master data of the laboratory measuring device 4 as well as the measurement data generated by the laboratory measuring device 4. The laboratory device data can also include status data or operating status data of the laboratory measuring device 4 which indicate a current operating status and/or operating mode of the laboratory measuring device 4.

The laboratory device data of a laboratory measuring device 4 contain e.g. information regarding the material sample measured by it, in particular a material sample ID (Sample ID). Further material sample information data include e.g. the density of the material sample P, its mass as well as its volume. The master data of the laboratory measuring device 4 include e.g. a serial number of the laboratory measuring device 4 and a type designation of the laboratory measuring device 4. The status data can indicate e.g. the test conditions. The measurement data generated by the laboratory measuring device 4 can relate to various measurement parameters, e.g. a measured temperature, a heat flux (W/m²), a thermal conductivity (W/mK), a measured thermal resistance, a measured temperature gradient, a measurement duration or a pressure (kPa). The number and type of measured parameters can vary greatly between different laboratory measuring devices 4. The different types of laboratory measuring devices 4 of a laboratory measuring device operator LMGB comprise laboratory measuring devices 4 of different laboratory measuring device producers and/or different laboratory measuring device types. The output data formats of the laboratory measuring devices 4 preferably have structured data sets which include data fields, wherein a data field can be provided for each measurement parameter and/or each master data type. The mapping information of a laboratory measuring device operator LMGB is stored in the configurable mapping table MT of the laboratory measuring device operator LMGB. The mapping table MT of the laboratory measuring device operator LMGB allocates to each data field of an output data format a data field of a target data format of the laboratory information and management system LIMS of the laboratory measuring device operator LMGB. This is illustrated by way of example also in FIGS. 4, 5. The Mapping Engine 2A outputs the converted laboratory device data in the target data format via the cloud interface 2D. The cloud interface 2D forms a logical interface at transport level to the LIMS systems. The converted laboratory device data are written in the target data format to the laboratory database 6-i of the laboratory information and management system 5-i of the laboratory measuring device operator LMGB. For this purpose, the converted laboratory device data are automatically transmitted via the cloud or data network 3 of the system 1 to a target address of the laboratory information and management system 5-i of the respective laboratory measuring device operator LMGB and are stored at this location in the associated laboratory database 6-i for further evaluation. In one possible embodiment, the conversion apparatus 2 receives reports of the various laboratory measuring devices 4 which include the laboratory device data of the laboratory measuring devices 4 in structured form in an output data format. The conversion apparatus 2 is able to read-in and interpret various output data formats, in particular file formats. These laboratory device data included within the received measuring device report are transferred automatically and in standardised form to the LIMS preferably via the API of the laboratory information and management system LIMS. The mapping engine 2A of the conversion apparatus 2 has e.g. a logic which maps the received laboratory device data to the data structure of the laboratory information and management system (LIMS) 5 of the laboratory measuring device operator LMGB.

In one possible embodiment, the mapping information or mapping configuration data included in the mapping table MT of the laboratory measuring device operator LMGB are configured in a configuration routine. In a further possible embodiment, the mapping table MT is automatically updated during operation of the laboratory measuring devices 4 by its laboratory measuring device operator LMGB. In a preferred embodiment of the system 1 in accordance with the invention, the mapping of the data fields is effected bidirectionally, i.e. in both directions. The laboratory device data stored in the target data format of the laboratory information and management system (LIMS) 5 of the laboratory measuring device operator LMGB in the laboratory database 6-i can be converted back to the output data format of the laboratory measuring device 4 according to the mapping information stored in the mapping table MT of the laboratory measuring device operator LMGB. In one possible embodiment, this can be effected in response to a back-conversion control command. The conversion apparatus 2 is able to process a large number of different output and target data formats. The data formats include in particular a CSV data format, a JSON data format and an XML data format. The file format CSV (Comma-Separated Values) describes the structure of a text file for storing or exchanging simply structured data separated e.g. by commas. The use of an XML file allows the use of complex data structures. XML files can be used to store data and formatting together with raw values or raw data of the measurements. Owing to the flexibility of the XML data format, additional information can be stored with the measurement data in a structured manner. These XML files can be opened and edited in text editors and XML-enabled browsers. Tags serve to describe the file structures. While XML files require a relatively large amount of storage space, Technical Data Management Streaming TDMS files only take up a small amount of storage space because they are in a binary data format. TDMS files have a file header, in which descriptions or additional information can be stored together with the laboratory device data. In addition, the data formats can include an INI data format, an ODS data format, an ODT data format, an RSS data format, an RDF data format, a TXT data format and a JSON data format. These data formats are machine-readable. In one possible embodiment, a sample number of the material sample is recorded during a measurement with the laboratory measuring device 4. This is effected e.g. by manually inputting a sample number or via a barcode reader of the laboratory measuring device 4. During a measuring procedure, the measurement results can be collected and exported with the aid of control software of the laboratory measuring device 4 and can be stored in an export file in an output data format. The export file is transmitted in the output data format from the laboratory measuring device 4 to the conversion apparatus 2 via a data interface. In one possible embodiment, the export file is transmitted to a configurable network address of the conversion apparatus 2. After importing the transmitted file, the conversion apparatus 2 can automatically convert the output data format of the laboratory measuring device 4 into the target data format of the laboratory information and management system LIMS of the laboratory measuring device operator LMGB. For this purpose, the mapping table MT of the recognised or detected laboratory measuring device operator LMGB which is stored in the configuration memory 2B is used by the mapping engine 2A. In one possible embodiment, the laboratory measuring device data are transmitted to the conversion apparatus 2 by wire. Alternatively, the laboratory measuring device data can also be communicated wirelessly from a laboratory measuring device 4 to the conversion apparatus 2. In one possible embodiment, the laboratory device data provided by the laboratory measuring device 4 are transmitted in the output data format as user data in data packets. The data packets contain the laboratory device data as payload data. For packet transmission of the laboratory device data, in an alternative embodiment the laboratory device data can also be transmitted in a data stream from the laboratory measuring devices 4 to the conversion apparatus 2 and from this location can be transmitted in converted form to the laboratory information and management systems LIMS. In a preferred embodiment, during an operation of a laboratory measuring device 4 of a laboratory measuring device operator LMGB, a bidirectional communication link exists between the laboratory measuring device 4 of the laboratory measuring device operator LMGB and the laboratory information and management system LIMS 5-i of the laboratory measuring device operator LMGB. The bidirectional communication link can be established indirectly via the conversion apparatus 2 or alternatively directly between the laboratory measuring device 4 and the LIMS of the laboratory measuring device operator LMGB. The bidirectional communication link is used for protocol-independent communication and for transmitting laboratory device data to the laboratory information and management system (LIMS) 5 of the laboratory measuring device operator LMGB and for transmitting control commands CMD from the laboratory information and management system (LIMS) 5 of the laboratory measuring device operator LMGB to its laboratory measuring devices 4. FIG. 1 schematically shows the transmission of control commands CMD (Commands) to the laboratory information and management system (LIMS) 5-i of the laboratory measuring device operator LMGB via the data network or the cloud 3 to the conversion apparatus 2 and from there to the respective laboratory measuring devices 4-i of the laboratory measuring device operator LMGBᵢ. In one possible embodiment, the laboratory device data and the control commands CMD are exchanged via the bidirectional communication link existing between the laboratory information and management system (LIMS) 5 and the laboratory measuring device 4 in real time. In this embodiment, the laboratory information and management system (LIMS) 5 of the laboratory measuring device operator LMGB has real-time data and can thus influence the measurement during a measuring procedure carried out by the laboratory measuring device 4. In order to be able to influence the real-time measuring procedure, the laboratory information and management system (LIMS) 5 of the laboratory measuring device operator LMGB can communicate corresponding control commands or commands CMD to the conversion apparatus 2 and/or to the laboratory measuring device 4-i of the laboratory measuring device operator LMGB. These control commands CMD can include e.g. measurement specifications for performing a measurement on a material sample. For example, desired or limit values for taking a material sample measurement can be transmitted to the laboratory measuring device 4. These measurement specifications can be adjusted dynamically during the measuring procedure with the aid of the transmitted measurement control commands CMD. In general, the measurement specifications are device-dependent. For example, the amount of flushing gas used by DSC devices from different producer differs. The amount of material required for a material sample also depends on the type of device and/or device producer. The control commands CMD can also include conversion control commands. For example, the laboratory information and management system (LIMS) 5-i can control in which target data format it wants to receive the laboratory measuring device data of the laboratory measuring device 4 or which objective is pursued with a measurement. The control commands CMD of the laboratory information and management system (LIMS) 5 which are received by the conversion apparatus 2 can also change the content of the mapping table MT stored in the configuration data memory 2B of the conversion apparatus 2 and can thus influence the mapping between the data fields. It is therefore also possible for the laboratory information and management system (LIMS) 5 of the laboratory measuring device operator LMGB to update the mapping table MT of the laboratory measuring device operator LMGB during the ongoing operation of the laboratory measuring devices 4. With the aid of the system 1 in accordance with the invention, as illustrated in FIG. 1, the exchange or replacement of a laboratory measuring device 4 by another laboratory measuring device 4 is simplified. It is also possible to automatically update inventory data of a laboratory measuring device 4. When a first laboratory measuring device 4 is replaced by a second laboratory measuring device 4′, the laboratory device data of the first original laboratory measuring device 4 which are stored in the laboratory database 6-i of the laboratory information and management system (LIMS) 5-i of the laboratory measuring device operator LMGB in the target data format are preferably seamlessly updated automatically with laboratory device data of the second new laboratory measuring device 4 in the target data format. This enables the laboratory measuring device operator LMGB to replace an old laboratory measuring device 4 of a specific laboratory measuring device producer and/or a specific laboratory measuring device type with a second new laboratory measuring device 4′ of another laboratory measuring device producer and/or another laboratory measuring device type without losing access to inventory data. In one possible embodiment, the laboratory information and management system (LIMS) 5 of the laboratory measuring device operator LMGB can determine or ascertain the respective utilisation of the various laboratory measuring devices 4 of the laboratory measuring device operator LMGB on the basis of the converted laboratory device data which are available in the target data format of the laboratory information and management system LIMS and are written to the laboratory database 6-i of the laboratory information and management system 5-i. Based on the ascertained individual utilisations (e.g. number of material samples per time period) of the various laboratory measuring devices 4-i of the laboratory measuring device operator LMGB, the laboratory information and management system 5-i of the laboratory measuring device operator LMGB can then increase the total utilisation of the laboratory measuring devices 4-i of the laboratory measuring device operator LMGB by transmitting corresponding control commands CMD to the laboratory measuring devices 4-i of the laboratory measuring device operator LMGB. The laboratory measuring devices 4-i of the laboratory measuring device operator LMGB comprise in particular material testing measuring devices for performing measurements on material samples P which are heated e.g. in a heating oven 4A of a laboratory measuring device 4. The measurement data generated by sensors in this process are transmitted in a defined output data format via the data interface of the laboratory measuring device 4-i to the conversion apparatus 2 and at this location are automatically converted into the target data format of the laboratory information and management system 5-i of the laboratory measuring device operator LMGB. In one possible embodiment, each laboratory measuring device 4-i can measure one or more material samples P simultaneously. If the laboratory information and management system 5-i of the laboratory measuring device operator LMGB recognises on the basis of the measurement data available in the target data format that a laboratory measuring device 4-i of a laboratory measuring device operator LMGB is completely utilised, while another laboratory measuring device 4-i of the same laboratory measuring device operator LMGB still has free capacities, material samples P can be automatically diverted to the laboratory measuring device which is not yet completely utilised, so that the total utilisation of the laboratory measuring devices 4-i of the laboratory measuring device operator LMGB is automatically increased. In one possible embodiment, the redistribution or redirection of material samples P can also be effected in real time for laboratory measuring devices 4, which are not yet completely utilised, with the aid of control commands CMD. In one possible embodiment, the conversion apparatus 2 illustrated in FIG. 3 provides a virtualisation layer. In this case, the conversion apparatus 2 creates a virtual group of laboratory measuring devices of the same type from a connected group of laboratory measuring devices 4-i of a laboratory measuring device operator LMGB, irrespective of the producer, for capacity and order planning by the laboratory information and management system LIMS of the laboratory measuring device operator LMGB.

In one possible embodiment of the system 1 in accordance with the invention, a universal uniform target data format is provided which enables the comparability of laboratory device data from laboratory measuring devices 4-i of different laboratory measuring device operators LMGB. In one possible embodiment, the conversion apparatus 2 converts the received laboratory device data of a laboratory measuring device 4-i of a laboratory measuring device operator LMGB not only into the specified target data format of the laboratory information and management system (LIMS) 5-i of the relevant laboratory measuring device operator LMGBᵢ, but additionally into a further universal target data format of the system 1 which is preferably supported by all incorporated laboratory information and management systems 5-i of the various laboratory measuring device operators LMGBᵢ. The laboratory device data stored in the uniform or comparison target data format in the laboratory databases 6-i of the various laboratory information and management systems 5-i allow various laboratory measuring devices 4-i to be compared in terms of their efficiency or performance and/or measuring accuracy. For example, a material sample P can be divided and measured in parallel by means of two laboratory measuring devices 4-i of the same type but different producers and the resulting laboratory measuring device data can be automatically compared with each other by means of an analysis-computing unit of the system 1. This allows e.g. the automatic calibration of a laboratory measuring device 4 by means of a reference laboratory measuring device.

FIG. 2 shows a flow diagram of one possible embodiment of the computer-implemented method in accordance with the invention for the efficient operation of laboratory measuring devices 4 of an LMGB, in particular for increasing utilisation of laboratory measuring devices 4 of the laboratory measuring device operator LMGB.

In the flow diagram illustrated in FIG. 2, the computer-implemented method includes essentially three main steps S1, S2, S3.

In a first step S1, laboratory device data are received from a laboratory measuring device 4-i of the laboratory measuring device operator LMGB in an output data format.

In a further step S2, the received laboratory device data are automatically converted into a target data format of the laboratory information and management system (LIMS) 5 of the laboratory measuring device operator LMGB. In this process, data fields of the output data format are automatically mapped or converted to data fields of the target data format according to mapping information stored in the mapping table MT of the laboratory measuring device operator LMGB.

In a further step S3, the converted laboratory device data in the target data format are written to a laboratory database 6 of the laboratory information and management system (LIMS) 5 of the laboratory measuring device operator LMGB, wherein the laboratory measuring devices of the laboratory measuring device operator (LMGB) communicate with the laboratory information and management system, LIMS, (5) of the laboratory measuring device operator (LMGB) to increase the total utilisation of the laboratory measuring devices (4) of the laboratory measuring device operator (LMGB).

The computer-implemented method can be implemented as middleware.

In an optional step S4, the utilisation of the laboratory measuring devices 4-i can be determined or ascertained on the basis of the laboratory device data available in the target data format of the laboratory information and management system LIMS of the laboratory measuring device operator LMGB. The utilisation of a laboratory measuring device 4 can include the number or quantity of material samples P measured per time period by means of a laboratory measuring device 4.

In one possible embodiment, the laboratory measuring devices 4-i can be controlled by means of the laboratory information and management system (LIMS) 5 of the laboratory measuring device operator to increase the total utilisation of the laboratory measuring devices 4-i of the laboratory measuring device operator LMGB in an optional step S5. For this purpose, the laboratory information and management system (LIMS) 5 of the laboratory measuring device operator LMGB outputs control commands CMD which are communicated to the laboratory measuring devices 4-i of the laboratory measuring device operator LMGB directly via the cloud 3 and/or indirectly via the conversion apparatus 2. The total utilisation indicates the total quantity or total number of all material samples P measured by the laboratory measuring devices 4 of the laboratory measuring device operator LMGB per time period (e.g. per day).

FIG. 4 shows by way of example the mapping of laboratory device data in the method in accordance with the invention. In the illustrated example, a laboratory measuring device LMGₓ outputs laboratory device data in structured form in an output data format. In the illustrated example, the output data format includes seven different data fields for different laboratory device data. In the illustrated example, the laboratory device data include measurement data on the one hand and master data of the laboratory measuring device on the other hand. In the illustrated example, the output data format of the laboratory measuring device 4 includes various data fields, namely a first data field for temperature measurement data T, a second data field for the measurement duration Δt, a third data field for the material M of the material sample, a fourth data field for the mass m of the material sample P, a fifth data field for the date D of the measuring procedure, a sixth data field for a time t of the measuring procedure, and a seventh data field LMG-ID for identifying the laboratory measuring device. In the example illustrated in FIG. 4, the laboratory measuring device LMGₓ is operated by a laboratory measuring device operator LMGB which uses the laboratory information and management system LIMS_{A}. This laboratory information and management system LIMS_{A} uses a data structure with likewise seven data fields as the target data format. The target data format includes a first data field for the mass m of the material sample, a second data field for the device ID (LMG-ID) of the laboratory measuring device, a third data field for the time t of the measurement, a fourth data field for the date D of the measurement, a fifth data field for the temperature T of the measurement, a sixth data field for the duration Δt of the measurement and a seventh data field M for the material of the material sample. According to the mapping information stored in the mapping table MT of the laboratory measuring device operator LMGB of the laboratory measuring device LMGₓ, the data contained in the various data fields are mapped, as illustrated schematically in FIG. 4. For example, the temperature measurement data T located in the first data field of the output data format are mapped to the fifth data field of the target data format of the laboratory information and management system LIMS_{A}. The data stored in the second data field of the output data format which indicate the duration Δt of the measuring procedure carried out are mapped to the sixth data field of the target data format in the illustrated example etc. In a preferred embodiment, the mapping and/or reconversion of the information or data stored in the various data fields is effected bidirectionally, as illustrated in FIG. 4. The mapping relates to the allocation, as illustrated in FIG. 4. The conversion or reconversion relates to a change in the data format or the language or syntax, with which the measurement data are made available by the device. The mapping and conversion can be performed independently of one another. Alternatively, the mapping and the conversion can also be performed simultaneously in one step.

As illustrated in FIG. 5, each laboratory measuring device LMGₓ, LMG_{y}, LMG_{z} can communicate comparable measurement parameters or information in different data fields of an output data format. For example, the temperature data T are communicated in the first data field in the first laboratory measuring device LMGₓ, in the ninth data field in the second laboratory measuring device LMG_{y} and in the third data field of the output data format in the third laboratory measuring device LMG_{z}. In the same way, measurement parameters or master data information can be transmitted or stored in different data fields of a target data format of a laboratory information and management system LIMS. For example, in the example illustrated in FIG. 5, the material information M is transmitted in the seventh data field of the target data format in the first laboratory information and management system LIMS_{A}, in the first data field of the target data format in the second laboratory information and management system LIMS_{B} and in the fifth data field of the target data format in the third laboratory information and management system MSc. The number of data fields in the output and target data formats can vary depending on the application. Not all data fields of a data format have to be filled.

For instance, in the example illustrated in FIG. 5, the laboratory measuring device LMGₓ is used by a laboratory measuring device operator LMGB which has the laboratory information and management system LIMS_{A}, the temperature information T contained in the first data field of the output data format of the laboratory measuring device LMGₓ is mapped or copied to the fifth data field of the output data format of the laboratory information and management system LIMS_{A}. If, however, the same laboratory measuring device LMG is used by another laboratory measuring device operator LMGB which has the second laboratory information and management system LIMS_{B}, the temperature data T contained in the first data field of the output data format are mapped or copied to the eighth data field of the target data format of the second laboratory information and management system LIMS_{B}. The system 1 in accordance with the invention is thus suitable for any laboratory measuring devices LMG and different laboratory information and management systems LIMS. By adapting the mapping table MT accordingly, the system 1 in accordance with the invention also makes it possible for a laboratory measuring device operator LMGB to change seamlessly between various laboratory information and management systems LIMS.

FIG. 6 shows a possible application example of the system 1 in accordance with the invention for the efficient operation of laboratory measuring devices 4, in particular for increasing the utilisation of laboratory measuring devices 4. In the application example illustrated in FIG. 6, the laboratory measuring device 4 of the system 1 is coupled to a production plant 7 having various production stages 8. In the production stage 8, a material sample Pj is taken manually or automatically and is placed in a heating oven 4A of the laboratory measuring device 4. The laboratory measuring device 4 has a plurality of sensors 4B-1, 4B-2, 4B-n for different measurement parameters, e.g. measurement temperature T, material mass or material expansion. These measurement data or sensor data are written by a processor or controller 4C of the laboratory measuring device 4 to an output file which is output via a data interface 4D of the laboratory measuring device 4 to the conversion apparatus 2 via the cloud or the data network 3. The output or export file has an output file format. The controller 4C can perform a measurement sequence control and activate e.g. a heating element 4E of the laboratory measuring device 4. Via a user interface 4F, a user of the laboratory measuring device 4 has the option of setting parameters of the measuring procedure or making input commands. Furthermore, measurement data can be displayed to the user via the graphical user interface 4F of the laboratory measuring device 4. The measurement data available in the output data format can additionally be stored or intermediately stored in a local data memory 4G of the laboratory measuring device 4. The controller 4C of the laboratory measuring device 4 can be connected to a plant controller 9 of the production plant 7 via a further data interface 4H. In one possible embodiment, this plant controller 9 generates control signals CRTL for various production stages 8 of the production plant 7 in dependence upon the laboratory device data available in the output data format, as illustrated schematically in FIG. 6. If e.g. the laboratory device data of the laboratory measuring device 4 indicate that the material sample P which is located in the heating oven 4A and was taken from the production stage 8j does not offer sufficient quality, the plant controller 9 can activate the upstream production stages of the production plant 7 in order then to produce sufficient quality. In the illustrated exemplified embodiment, an analysis unit of the plant controller 9 evaluates the laboratory device data provided by the laboratory measuring device 4 in the output data format and activates one or more production stages 8 of the production system 7 in dependence upon the analysis result. In a further possible embodiment, the laboratory information and management system LIMS of the laboratory measuring device operator LMGB of the laboratory measuring device 4 can generate control commands or commands CMD which are transmitted via the laboratory measuring device 4 to the plant controller 9 of the production plant 7 in order to activate the production plant 7 accordingly. In an alternative embodiment, the laboratory information and management system LIMS of the laboratory measuring device operator LMGB can directly activate the plant controller 9 of the production plant 7. For this purpose, the plant controller 9 is connected to the data network or the cloud 3 of the system 1 via an interface. In one possible embodiment, the data exchange for communicating the control commands CMD is effected in real time during the production procedure. The network infrastructure 3 of the system 1 in accordance with the invention can use different network protocols, in particular TCP/IP. Via the network infrastructure 3 of the system 1, the laboratory measuring devices 4 can be connected as clients to a server which executes a server application. In one possible embodiment, this server application can execute one or more main steps of the computer-implemented method illustrated in FIG. 2. The server application can also be distributed to a plurality of servers which can be operated internally or even externally by IT service providers or in the cloud. In a further possible embodiment, the laboratory measuring device 4 illustrated in FIG. 6 has a further data interface for connecting a local computer or PC. This PC preferably offers the user of the LIMS application a graphical user interface. The LIMS application can be operated in parts or completely via a web browser. The LIMS application can also be provided in part with the aid of mobile devices or tablets. The system 1 in accordance with the invention offers industry-wide networking of different types of laboratory measuring devices 4 of different laboratory measuring device operators LMGB to different laboratory information and management systems LIMS. Manual data management is avoided and capacity planning for the various laboratory measuring devices 4 is facilitated. The system 1 in accordance with the invention also enables benchmarking of the performance or efficiency of different laboratory measuring devices 4. With the aid of the system 1 in accordance with the invention, laboratory measuring devices 4 of different types or from different manufacturers can be exchanged in a simple manner by the laboratory measuring device operator LMGB without losing access to existing inventory data or measurement data or without the need to have to manually adjust these inventory data. The system 1 in accordance with the invention is also real-time enabled, i.e. capacity planning or utilisation adjustment can be effected in real time. The conversion apparatus 2 preferably provides a bidirectional interface between the laboratory measuring devices 4 on the one hand and the laboratory information and management systems (LIMS) 5 on the other hand. The laboratory information and management system (LIMS) 5 offers the user a broad spectrum of functions, in particular for measurement data acquisition, support in sample distribution and sample processing, order registration and sample registration, release of test results as well as measurement data evaluation and calculation of analysis results.

In a preferred embodiment, the configuration apparatus 2 can be installed on a server of a cloud platform. This offers greater availability and safeguarding against failure through a redundant server system. Furthermore, the laboratory database 6 of the laboratory information and management system (LIMS) 5 can be accessed from anywhere. Data backup can be effected with a high frequency, e.g. by the second. With the aid of the server system of the cloud platform, high performance and efficiency can be achieved in the evaluation and management of laboratory device data. In one possible embodiment, the laboratory database 6 of the laboratory information and management system 5 is structured in such a way that all components are redundantly distributed in various independent availability zones. This reduces the probability of a failure of the laboratory database 6 of the laboratory information and management system 5.

Further embodiments of the system 1 in accordance with the invention are possible. For example, in one possible embodiment, the laboratory device data are transmitted in encrypted form. In one possible embodiment variation the laboratory device data can also be encoded in order to reduce transmission errors.

1. Computer-implemented method for the efficient operation of laboratory measuring device (4) of a laboratory measuring device operator (LMGB), comprising the steps of:
receiving (S1) laboratory device data which are output by different types of laboratory measuring devices (4) of the laboratory measuring device operator (LMGB) in different output data formats,
converting (S2) the received laboratory device data into at least one specified target data format of a laboratory information and management system, LIMS, (5) of the laboratory measuring device operator (LMGB),
wherein data fields of the output data formats are automatically converted to data fields of the target data format according to mapping information stored for the laboratory measuring device operator (LMGB),
writing (S3) the converted laboratory device data in the target data format to a laboratory database (6) of the laboratory information and management system LIMS, (5) of the laboratory measuring device operator (LMGB),
wherein the laboratory measuring devices of the laboratory measuring device operator (LMGB) communicate with the laboratory information and management system LIMS, (5) of the laboratory measuring device operator (LMGB) to increase the total utilisation of the laboratory measuring devices (4) of the laboratory measuring device operator (LMGB).

2. Computer-implemented method as claimed in claim 1,
wherein the respective utilisation of a laboratory measuring device (4) of the laboratory measuring device operator (LMGB) is determined (S4) by the laboratory information and management system, LIMS, (5) of the laboratory measuring device operator (LMGB) on the basis of the laboratory device data which are converted into the target data format of the laboratory information and management system, LIMS, (5) of the laboratory measuring device operator (LMGB) and are written to the laboratory database (6) of the laboratory information and management system, LIMS, (5).

3. Computer-implemented method as claimed in claim 2,
wherein the laboratory measuring devices (4) of the laboratory measuring device operator (LMGB) are controlled (S5) by the laboratory information and management system, LIMS, (5) of the laboratory measuring device operator (LMGB) in dependence upon the determined utilisations of the laboratory measuring device (4) for controlling the total utilisation of the laboratory measuring devices of the laboratory measuring device operator (LMGB).

4. Computer-implemented method as claimed in any one of claims 1 to 3,
wherein the laboratory device data of the laboratory measuring device (4) include master data of the laboratory measuring device (4), measurement data of the laboratory measuring device (4) and/or status data of the laboratory measuring device (4).

5. Computer-implemented method as claimed in any one of claims 1 to 4,
wherein the different types of laboratory measuring devices (4) of the laboratory measuring device operator (LMGB) comprise laboratory measuring devices (4) of different laboratory measuring device producers and/or different laboratory measuring device types, which provide master data of the laboratory measuring device (4) and generate measurement data and output same in a defined output data format of the laboratory measuring device (4).

6. Computer-implemented method as claimed in any one of the preceding claims 1 to 5,
wherein the output data formats of the laboratory measuring devices (4) include structured data sets with data fields for different measurement parameters and/or master data types.

7. Computer-implemented method as claimed in any one of the preceding claims 1 to 6,
wherein the mapping information of the laboratory measuring device operator (LMGB) are stored in a configurable mapping table (MT) which allocates to each data field of an output data format a data field of a target data format of the laboratory information and management system, LIMS, (5) of the laboratory measuring device operator (LMGB).

8. Computer-implemented method as claimed in any one of the preceding claims 1 to 7,
wherein, during the conversion procedure for converting the received laboratory device data into at least one specified target data format of the laboratory information and management system, LIMS, (5) of the laboratory measuring device operator (LMGB), the laboratory device data contained within a data field of the output data format are imported into the allocated data field of the target data format of the laboratory information and management system, LIMS, (5) of the laboratory measuring device operator (LMGB).

9. Computer-implemented method as claimed in claim 7 or 8,
wherein the mapping information contained in the mapping table (MT) of the laboratory measuring device operator (LMGB) is configured in a configuration routine and/or is automatically updated during the operation of the laboratory measuring devices (4) of the laboratory measuring device operator (LMGB).

10. Computer-implemented method as claimed in any one of the preceding claims 1 to 9,
wherein the laboratory device data of the laboratory measuring device (4) which are stored in the target data format of the laboratory information and management system, LIMS, (5) of the laboratory measuring device operator (LMGB) in the laboratory database (6) are converted, in response to a conversion control command, back to the output data format of the laboratory measuring device (4) according to the mapping information stored in the mapping table (MT) of the laboratory measuring device operator (LMGB).

11. Computer-implemented method as claimed in any one of the preceding claims 1 to 10,
wherein the laboratory measuring device (4) of the laboratory measuring device operator (LMGB) has a material testing measuring device for performing measurements on material samples (P), wherein measurement data generated by a measurement are output in an output data format via a data interface of the laboratory measuring device (4).

12. Computer-implemented method as claimed in claim 11,
wherein, during the operation of the laboratory measuring device (4), a controller (4C) of the laboratory measuring device (4) of the laboratory measuring device operator (LMGB) receives measurement control commands with measurement specifications for performing measurements on material samples (P) from the laboratory information and management system, LIMS, (5) of the laboratory measuring device operator (LMGB).

13. Computer-implemented method as claimed in any one of the preceding claims 1 to 12,
wherein the laboratory measuring device (4) of the laboratory measuring device operator (LMGB) as a client provides laboratory device data in an output data format of the laboratory measuring device (4), which are transmitted as user data in data packets via a data network (3) to a conversion apparatus (2) implemented on a local or remote server, wherein the server executes a server application which automatically converts the received laboratory device data of the laboratory measuring device (4) into the specified target data format of the laboratory information and management system, LIMS, (5) of the laboratory measuring device operator (LMGB) according to the mapping information stored for the laboratory measuring device operator (LMGB).

14. Computer-implemented method as claimed in any one of the preceding claims 1 to 13,
wherein, when a first laboratory measuring device (4) of a specific laboratory measuring device producer (LMGB) and/or of a specific laboratory measuring device type is replaced by another second laboratory measuring device (4′) of another laboratory measuring device producer (LMGB′) and/or of another laboratory measuring device type, the laboratory device data of the first laboratory measuring device (4) which are stored in the laboratory database (6) of the laboratory information and management system, LIMS, (5) of the laboratory measuring device operator (LMGB) in the target data format are seamlessly updated automatically with laboratory device data of the second laboratory measuring device (4′) in the target data format.

15. Computer-implemented method as claimed in any one of the preceding claims 1 to 14, wherein, during the operation of a laboratory measuring device (4) of a laboratory measuring device operator (LMGB), a bidirectional communication link exists between the laboratory measuring device (4) of the laboratory measuring device operator (LMGB) and the laboratory information and management system, LIMS, (5) of the laboratory measuring device operator (LMGB) for the purpose of exchanging laboratory device data and control commands.

16. Computer-implemented method as claimed in claim 15,
wherein the laboratory device data and the control commands are exchanged in real time via the bidirectional communication link between the laboratory measuring device (4) and the laboratory information and management system, LIMS, (5).

17. System (1) for the efficient operation of laboratory measuring devices (4) of a laboratory measuring device operator (LMGB), the system (1) comprising:
laboratory measuring devices (4) of the laboratory measuring device operator (LMGB), which each provide laboratory device data in different output data formats;
a conversion apparatus (2) for converting the received laboratory device data into at least one specified target data format of a laboratory information and management system, LIMS, (5) of the laboratory measuring device operator (LMGB),
wherein data fields of the output data formats are automatically converted into data fields of the target data format according to mapping information stored for the laboratory measuring device operator (LMGB);
a laboratory database (6) of the laboratory information and management system, LIMS, (5) of the laboratory measuring device operator (LMGB), to which the laboratory device data which are converted into the target data format of the laboratory information and management system, LIMS, (5) of the laboratory measuring device operator (LMGB) are written; wherein the laboratory measuring devices of the laboratory measuring device operator (LMGB) communicate with the laboratory information and management system, LIMS, (5) of the laboratory measuring device operator (LMGB) to increase the total utilisation of the laboratory measuring devices (4) of the laboratory measuring device operator (LMGB).

18. System as claimed in claim 17,
wherein the laboratory information and management system, LIMS, (5) of the laboratory measuring device operator (LMGB) determines the respective utilisation of the laboratory measuring devices (4) of the laboratory measuring device operator (LMGB) on the basis of the laboratory device data, which are converted into the target data format of the laboratory information and management system, LIMS, (5) and are written to the laboratory database (6) of the laboratory information and management system, LIMS, (5), and controls the laboratory measuring devices (4) of the laboratory measuring device operator (LMGB) in order to increase the total utilisation of the laboratory measuring devices (4) of the laboratory measuring device operator (LMGB) by means of control commands (CMD) which the laboratory information and management system, LIMS, (5) of the laboratory measuring device operator (LMGB) transmits to the laboratory measuring devices (4) of the laboratory measuring device operator (LMGB).

19. Conversion apparatus (2) for automatically converting laboratory device data received in different output data formats into at least one specified target data format of a laboratory information and management system, LIMS, (5) of a laboratory measuring device operator (LMGB),
wherein data fields of the output data formats are automatically mapped to data fields of the target data format according to mapping information stored in a mapping table (MT) of the laboratory measuring device operator (LMGB).
